(19) United States
(12) Patent Application Publication
(10) Pub. No.: US 2024/0192200 A1
(43) Pub. Date: Jun. 13, 2024

(54) A PORTABLE DEVICE FOR TESTING AGRO-DAIRY BASED SAMPLES

(71) Applicant: Faunatech Solutions Private Limited, Mumbai (IN)

(72) Inventors: Rajat Pandya, Bangalore (IN); Sidhant Jena, Bangalore (IN); Sandhyaa Subramanian, Bangalore (IN); Rajashekar Reddy Palvalli, Bangalore (IN)

(73) Assignee: Faunatech Solutions Private Limited, Mumbai (IN)

(21) Appl. No.: 18/286,730

(22) PCT Filed: May 6, 2022
(86) PCT No.: PCT/IN22/50440

§ 371 Date: Oct. 12, 2023

(30) Foreign Application Priority Data
May 10, 2021 (IN) 202141021044

(51) Int. Cl. G01N 33/52; G01N 15/01; G01N 21/59; G01N 21/77; G01N 21/78; G16B 40/10

(52) U.S. Cl. CPC G01N 33/521; G01N 15/01; G01N 21/59; G01N 21/78; G16B 40/10; G01N 2021/7759; G01N 2201/0221; G01N 2333/904

(57) ABSTRACT

The invention relates to a portable device for testing agro-dairy based samples. The said device comprises an optical detector based on micro-spectroscopy. With the aid of an adaptor, the device is amenable to holding a colorimetric test strip, a concave shaped holder for a solid or semi-solid substance and/or a cuvette. The invention also describes a system of testing agro-dairy based samples comprising the said portable device and also a method of testing agro-dairy based samples using the said system.

### FIELD OF THE INVENTION

The present invention generally relates to a portable device for testing agro-dairy based samples. The invention also describes a system of testing agro-dairy based samples comprising the said portable device. The invention further discloses a method of testing agro-dairy based samples using the said system.

### BACKGROUND OF THE INVENTION

The agricultural sector and dairy sector often need to determine the quality of products like wine, beer, milk, juices, honey etc. These products are an important source of revenue for formers and are usually produced in large volumes but without appropriate quality checks.

Further, ascertaining the quality of products like milk is important for dairy formers as they receive revenue on the basis of milk components such as fat and protein and, commercially, milk of high nutritional content receives the best market price. Also, milk quality is a significant indicator of herd health and, for this, milk testing is invaluable. Milk testing is also useful to check for mastitis, an infection that affects the mammary glands in a milch animal's udder. Cows with mastitis produce lower quantities of milk and their milk also contains a large number of somatic cells (SCs).

Formers do not have easy access to testing laboratories or even the funds to regularly test their products to check for uniformity in quality, presence of contaminants or infections in milch animals. There is a pressing need for rapid testing and frequent screening for early detection of infected animals such that the infected animals can be promptly treated and so that their milk would no longer be pooled into the collection from other healthy animals thereby lowering the overall quality of the pooled milk. As mastitis can be inferred from high somatic cell count (SCC) in milk, there is a need for dairy formers to be able to do tests on the farm itself and obtain quick results.

There is a clear need for a single system to monitor herd health on the farm including screening for mastitis by measuring analytes such as SCC and also determining milk quality at field collection sites by measuring fats, solids-not-fat (SNF) content, lactate dehydrogenase (LDH), β-hydroxybutyrate (BHB), antibiotic residues and proteins in raw milk.

Apart from milk, there is a similar need for determining the quality of other agro-based and allied sector products like wine, beer, milk, juices, honey etc. There is a need for a portable testing device that does away with the need for separate lab testing, which involves a time lag in obtaining results and which presents a hurdle for remote rural locations lacking easily accessible laboratories.

Infrared spectroscopy is commonly used to ascertain fat and protein content in a sample while flow cytometry and direct microscopy are often used to determine SCC in milk samples. These ordinarily require trained lab technicians and follow a pay-per-test revenue model.

Research on the fabrication of miniaturized devices has focused on manufacturing with high precision and at low cost especially for emerging markets. There is a need for a miniaturized testing device which is easy to use even by an uneducated person, one that is amenable to a variety of testing requirements using a single device and also one that gives real-time results. The cost benefit to the farmer would also be significant in terms of doing away with payments for each test.

### SUMMARY OF THE INVENTION

According to an embodiment of the invention there is provided a portable device for testing agro-dairy based samples, said device comprising:
a. at least one optical detector based on micro-spectroscopy, said detector being optically aligned with at least one test sample and having sensitivity across a wavelength range encompassing reflectance or transmission wavelength of at least one test analyte of the test sample;
b. at least one adaptor holding
   i. at least one colorimetric test strip upon which is placed the at least one test sample comprising the at least one test analyte;
   ii. at least one concave shaped holder for holding the at least one test sample, said sample being solid or semi-solid; and/or
   iii. at least one cuvette for holding the at least one test sample further containing at least one test analyte;
said adaptor(s) for each of (i), (ii) and/or (iii) fitting into at least one socket on the device;
c. at least one light emitting diode (LED) light source capable of emitting a range of wavelengths;
d. a microcontroller for controlling the functioning of electronic components; and
e. a networking module for data transfer utilizing wireless technology.

According to another embodiment of the invention, there is provided a system of testing agro-dairy based samples comprising:
a. the portable testing device as described above;
b. a power source; and
c. a computing device remotely connected to the portable testing device, via the wireless technology of the networking module, said computing device having a computer program for monitoring, recording, storing, or analysis of spectral data, or a combination thereof, wherein the computing device has an electronic processor that executes the said computer program to process the said spectral data to provide a measurement value for the sample(s) tested.

According to yet another embodiment of the invention, there is provided a method of testing agro-dairy based samples using the system as described above, comprising the steps of:
a. powering on the portable testing device and remotely connecting the said device to the computing device;
b. inputting suitable information into the computer program of the computing device to enable the computer program to instruct the portable testing device to calibrate the LED light source(s) and the corresponding optical detector(s) in a specific way;
c. introducing the at least one test sample into the at least one cuvette, and/or onto the at least one colorimetric test strip, and/or onto the at least one concave holder, and inserting the corresponding adaptor(s) holding the said cuvette and/or the said test strip and/or the said concave holder into corresponding socket(s) on the device;
d. turning on the LED light source(s) to direct light towards the sample(s) thereby enabling the corresponding optical detector(s) to receive light reflected from or transmitted through the sample and communicate the same in the form of spectral data to the computing device via the wireless technology;
e. executing the computer program on the computing device to process the spectral data to provide a measurement value for the sample tested; and
f. reading a display screen on the remotely connected computing device showing the measured output data of reflectance or transmission of the sample tested.

In some instances, a reference sample or a blank is run prior to step (c). This is mainly done to account for the matrix or the solution in which the biomarker is present, so that the biomarker concentration can be calculated accurately using a simple model.

### BRIEF DESCRIPTION OF THE DRAWINGS

FIG. 1A is a diagrammatic representation of the portable testing device along with an adaptor for a colorimetric test strip placed alongside the device.

FIG. 1B is a diagrammatic representation of the portable testing device along with an adaptor for a colorimetric test strip inserted into the socket of the device.

FIG. 2A is a diagrammatic representation of the portable testing device along with an adaptor for a cuvette placed alongside the device.

FIG. 2B is a diagrammatic representation of the portable testing device along with an adaptor for a cuvette inserted into the socket of the device.

FIG. 2C is a diagrammatic representation of the portable testing device along with an adaptor for a cuvette inserted into the socket of the device, said adaptor accommodating a cuvette.

FIG. 3 is a schematic diagram of an off-set top sectional view of the portable testing device, according to an embodiment of the invention.

FIG. 4 is a diagrammatic representation of the bottom storage space of the portable testing device, according to an embodiment of the invention.

FIG. 5 is a diagrammatic representation of the portable testing device along with an adaptor holding a concave shaped holder for holding a solid or semi-solid sample substance to be tested.

FIG. 6 is a diagrammatic representation of the adaptor fused to the concave shaped holder, according to an embodiment of the invention.

FIG. 7 is a graph comparing the performance of the device according to an embodiment of the present invention versus the DeLaval Cell Counter for an SCC test.

FIG. 8 is a Receiver Operating Characteristic (ROC) curve for an SCC test using the device and method according to an embodiment of the present invention versus DeLaval Cell Counter (AUC of 0.98 for subclinical mastitis)

FIG. 9 is a graph representing the transmittance data collected by the device of the present invention for cow milk samples with increasing values of milk fat %.

### DETAILED DESCRIPTION OF THE INVENTION

In the following detailed description, reference is made to the accompanying drawings that form a part hereof. The embodiments of the invention are described in sufficient detail to enable those skilled in the art to practice the invention and it is understood that other embodiments may be utilized and that logical processual changes may be made without departing from the spirit or scope of the invention. To avoid detail not necessary to enable those skilled in the art to practice the embodiments described herein, the description may omit certain information known to those skilled in the art. The following detailed description is, therefore, not to be taken in a limiting sense and the scope of the illustrative embodiments are defined only by the claims appended hereinbelow.

The system of the invention comprises a portable testing device which functions as a handheld spectrometer to analyze both solid state chemistry platforms such as colorimetric test strips that react upon the application of various fluids by measuring reflectance, as well as analyze fluids directly by measuring transmission through the sample held in a cuvette. These fluids can be raw milk, beer, wine and other industrial extracts. The device can also measure the color of solid or semi-solid substances by reflectance measurements, and this is useful for small sized fruit, spices or produce whose ripening color determines the time of harvest.

The invention provides a portable, handheld testing device for measuring antibiotic residue, fats, hormones, enzymes, somatic cell counts, solids-not-fats, and proteins in raw milk, said device comprising a chemical sensor i.e. colorimetric test strip, a cuvette and a micro-spectrometer based optical detector. The device reads colorimetric test strips, reflectance of small sized farm produce and measures transmission through fluids, and interfaces with a smartphone through wireless low power Bluetooth (BLE) connectivity and/or WiFi connectivity.

It is known that colorimetric test strips react to fluid samples by a change in the color of the reaction pad. In the device of the invention, a micro-spectrometer sensor that is optically aligned with the reaction pad on the test strip then detects this change. The change in color corresponds to and is then mapped to the somatic cell count or to concentrations of one of several parameters such as antibiotic residue, LDH, fats and proteins among others.

Similarly, the color of solid or semi-solid substances including, but not limited to, small sized fruit, spices or other form produce, can be measured using the portable testing device rather than relying on subjective determinations based on human visual perceptions. By depending on the micro-spectrometric reflectance readings of the color of small sized produce, harvest days can be objectively determined.

Liquids are analyzed using the device by passing a light source through a cuvette containing a sample. An optical detector i.e. micro-spectrometer sensor that is optically aligned with the cuvette holding adaptor then detects the transmission spectrum which in-turn corresponds to and is mapped to concentrations of fats, SNF content, LDH, BHB, antibiotic residues and proteins. The device can also be used to test blood and urine samples.

In one embodiment of the invention, multiple colorimetric test strips can be inserted into the device when multiple sockets are provided on the device with corresponding multiple adaptors, LED light sources and respective optical detectors. Similarly, multiple types of samples can be tested on a single device using different adaptors to hold either a colorimetric test strip and/or a concave shaped holder and/or a cuvette if multiple sockets are provided on the same device with corresponding LED light sources and their respective optical detectors.

The end user will interact with the device through a computer program or an application (app) that will be installed on their mobile phones or computers. Thus, the app would function as the user interface for the end user to perform analyte testing by providing graphical instructions for each step of the process. At the end of testing, the app displays the reading of the parameter being tested, along with trending charts of previous tests conducted by the user. The app, thus, controls the device and processes the raw color sensor data that the device sends to compute the reading. Thus, the device is amenable to advances in technology as the computation, storing, and analysis of the data is done by a suitable app which can be updated from time to time.

The invention also provides a method for analyzing the chemical sensor signal, said method including algorithms to quantify SCC using both a chemical sensor, i.e. through reflectance, and a cuvette i.e. through transmission in a liquid product such as raw milk.

Preferably, the measured output data is stored, analyzed, computed, recorded or used to show trends in the case of the same analytes being tested, or the same parameter being determined, across several samples.

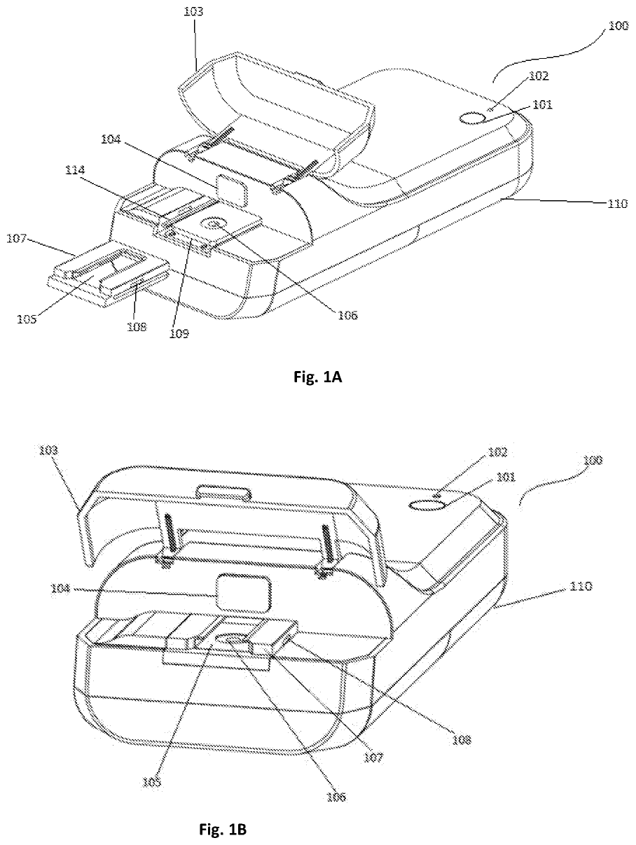

FIG. 1A is a diagrammatic representation of the top of the portable testing device (100) along with an adaptor (107) for a colorimetric test strip placed alongside the device. The device (100) has a soft power off/on button (101) and a corresponding LED indicator light (102) for the same. The device has a socket (109) for receiving the adaptor (107), said socket having guide rails (114) along which the adaptor (107) can be inserted into the device.

According to an embodiment of the invention, an adaptor for a colorimetric test strip has two slots for insertion of two colorimetric test strips (105, 108), each slot being of a different size to accommodate test strips of different sizes. The slots are positioned such that if two strips were simultaneously inserted into the respective two slots, the strips would be at right angles to each other. However, measurement of reflectance data is only possible if one inserts only one strip at a time in a single adaptor.

The device is housed in an enclosure or case, there being a separate modular cover (103) at the top of the device where the socket (109) for the adaptor (107, 111) is located, which modular cover can be independently opened or fastened shut, and there being a further separate cover (110) at the bottom of the device which can also independently be opened or fastened shut.

According to an embodiment of the invention, the device (100) also has an eject button (104) to release the top modular cover (103) such that one can replace a modular cover of one size with a modular cover of a different size, depending on the size of the adaptor being used for testing. Since this top part of the device enclosed by the modular cover (103) contains the strip/cuvette insertion area, in other words the rail (114) that guides the strip as it is inserted into the device, it allows the device to support strips and cuvettes of various materials or dimensions. This is a unique feature as compared to other analyzers available in the market and allows the device of the present invention to be quickly adapted to support nearly any colorimetric test strip and cuvette, as required, by simply making a new modular cover for the new strip or cuvette dimensions.

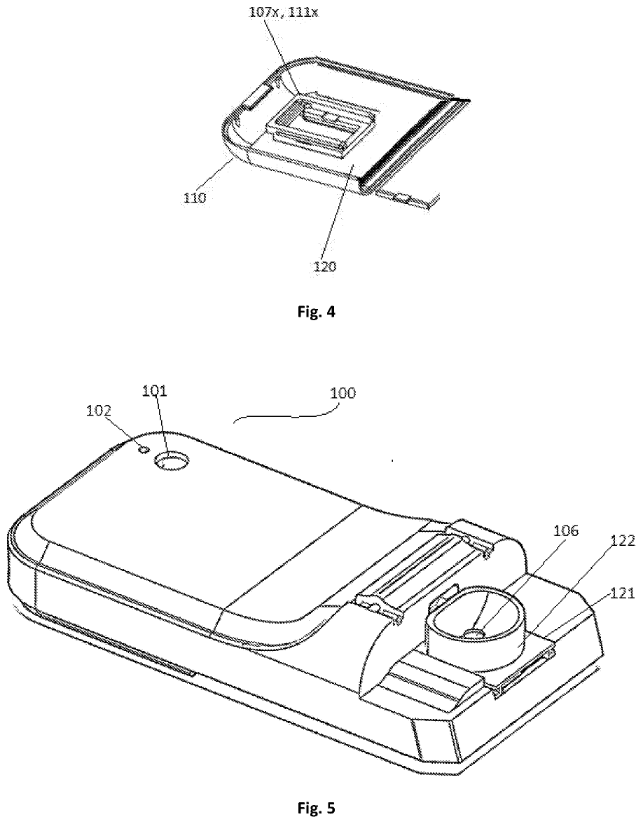

The bottom cover (110) and the storage space enclosed by the bottom cover are further described in FIG. 4.

An optical window (106) enables the light transmitted through a sample or reflected from a sample to pass through and be detected by the optical detector.

FIG. 1B is a diagrammatic representation of the top of the portable testing device (100) along with an adaptor (107) for a colorimetric test strip inserted into the socket of the device.

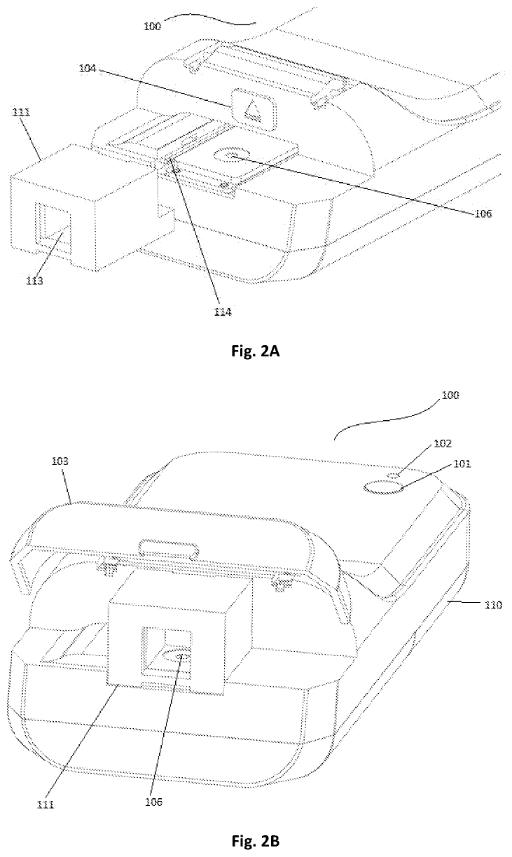

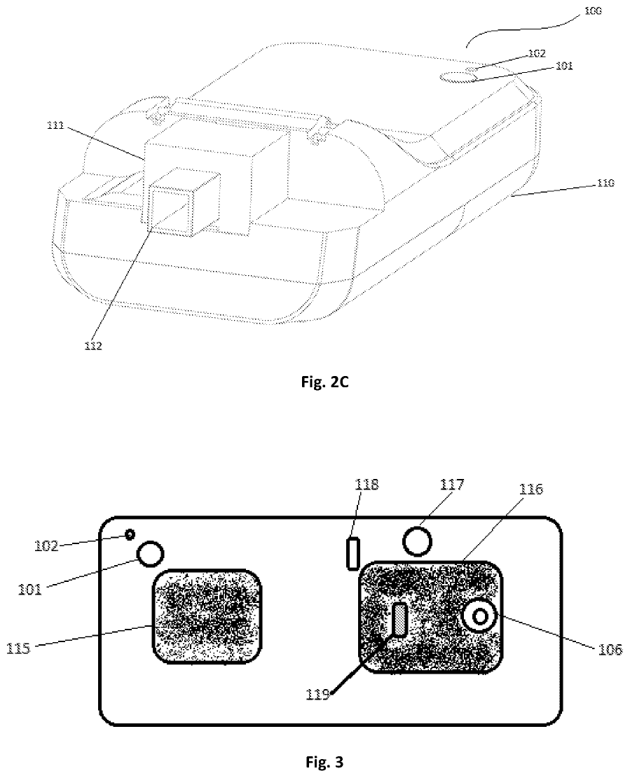

FIG. 2A shows the portable testing device (100) along with an adaptor (111) for a cuvette placed alongside the device while FIG. 2B shows the portable testing device along with an adaptor (111) for a cuvette inserted into the socket of the device.

FIG. 2C shows the portable testing device (100) along with an adaptor (111) for a cuvette inserted into the socket of the device, said adaptor accommodating a cuvette (112).

Each of the adaptors are custom designed for and connect the component that they hold i.e. test strip or holder for a solid/semi-solid substance or cuvette, as the case may be, to the main body of the device. At a given point of time, the device can determine reflectance of colour of one test strip or solid/semi-solid substance or transmission through the sample liquid held in one cuvette, but not all simultaneously.

According to an embodiment of the invention, the concave holder for solid or semi-solid substances is used to determine ideal colour of small sized form produce eg fruits like grapes, dates, olives etc, spices like cardamom etc for the purpose of colour measurement such that harvesting is done at the ideal time.

FIG. 3 is a schematic diagram of an off-set top sectional view of the portable testing device (100), according to an embodiment of the invention. Internally, the device (100) has a printed circuit board (PCB) micro-controller (115) which comprises a networking module which can be a Bluetooth module or a WiFi module or both. An optical block (116) comprises the optical detector (106) and the light source (119). A temperature sensor (117) serves to check the temperature of samples before taking readings as the device functions optimally within a specific temperature range i.e. 15 to 40° C.

A hard power on/off button (118) is also located internally. The LED light source (119) serves to emit light of a specific wavelength towards the sample being tested. The LED (119) can be on a separate board that couples to the optical block (116) in a way that illuminates the sample and minimizes stray or unwanted light.

FIG. 4 is a diagrammatic representation of the bottom storage space (120) enclosed by the bottom cover (110) of the portable testing device (100), according to an embodiment of the invention. The cover (110) at the bottom encloses a storage space (120) where it is possible to store a few more adaptors (107x, 111x) for the sake of convenience. In one embodiment of the invention, the bottom storage space can store two adaptors for colorimetric test strips, each adaptor having slots of different sizes to accommodate up to 4 different sizes of strips, i.e. two different sizes per adaptor. Thus, according to an embodiment of the invention, the device can allow for strips of 6 different sizes since it can store two different adaptors in the bottom storage space and can accommodate one adapter adjacent to the optical window where a sample is being tested. Alternatively, according to another embodiment of the invention, the device can store one cuvette adaptor and one test strip adaptor in the storage space of the case while another adaptor for a cuvette or for a test strip is simultaneously accommodated adjacent to the optical window (106) where a sample is being tested.

FIG. 5 is a diagrammatic representation of the portable testing device (100) along with an adaptor (121) holding a concave shaped holder (122) for holding a solid or semi-solid sample substance to be tested.

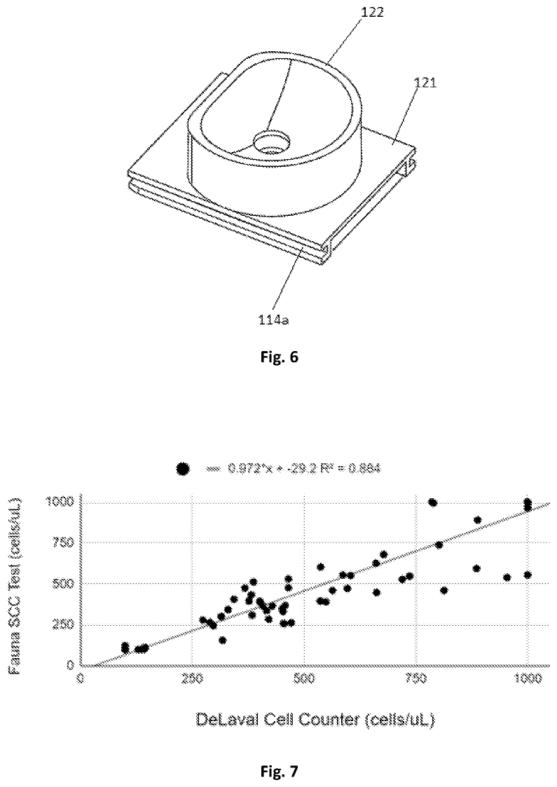

FIG. 6 is a diagrammatic representation of the adaptor (121) fused to the concave shaped holder (122), according to an embodiment of the invention. All adaptors have a groove (114a) to slide along the rails (114) on the device.

The device is affordable and versatile, especially in cost-sensitive emerging markets where the dairy industry is large and mostly comprising small formers who do not have access to quality testing infrastructure. In terms of cost, the farmer would make a one-time investment at the time of purchasing the device, and thereafter, testing costs would be practically negligible as only testing strips would involve a nominal cost. The versatility of the device is evident from it being able to support colorimetric test strips of different sizes, and cuvettes of varying dimensions, being made of different materials or being of varying thickness. Thus, the modular device design not only enables both reflectance and transmission measurements but also accommodates test strips of different dimensions as also different sizes of cuvettes.

The device itself does not have any intelligence. After an initial handshake it receives a configuration from the phone app instructing it to calibrate its optical system in a specific way. This includes the ID(s) of the LED(s) and micro-spectrometer to use i.e. the app determines the wavelength(s) used for the color analysis, the sampling period i.e. the time between successive sensor readings, and the pulse width modulation (PWM) duty cycle relating to the brightness of the LEDs. The device continuously sends raw data as measured by the color sensor to the phone app. The phone app actually does all of the data processing and analysis, and may reconfigure the LED blinking sequence of the device during the test if needed.

This system design provides several unique advantages when compared to similar products in the market. Firstly, it once again allows the user to add support to a new type of test strip, cuvette or chemistry by simply updating the phone app with new analysis algorithms via the usual app update channels (Google Play Store, Apple App Store, etc.) Secondly, this enables a very simple and cost-effective design of the device since it only requires minimal processing and computing capabilities as it is primarily controlled by the app.

The device of the invention is also amenable to analysis and calibration algorithms. By measuring the initial and final test strip at multiple wavelengths, one can correct for variations in manufacturing and activity to provide a more precise and accurate reading. Additionally, by measuring the signal over time, one can speed-up the measurement time by kinetically predicting the result without waiting for the end-point.

Other uses of the device would include testing blood and urine samples from animals and human beings, as also testing a range of samples including, but not limited to, fuels, solvents, paints, industrial effluents, dyes, and gemstones.

The following experimental examples are illustrative of the invention but not limitative of the scope thereof:

### Example 1: Accuracy Evaluation During Pilot

A DeLaval Cell Counter was used as a reference analyzer. Out of the 210 samples tested, 78 samples were referenced on the DeLaval Cell Counter. Holstein Fresians and other breeds were used to obtain samples for testing.

The samples were tested about three to six hours after milking from the cow. The SCC test using the device and method of the present invention showed good correlation versus the DeLaval Cell Counter with R²=0.88 and 94% and 86% of the samples agreeing with the DeLaval Cell Counter measurements in the healthy and mastitis-infected categories, respectively as shown in Table 1. In particular, Table 1 shows the agreement of the SCC values binned correctly by the SCC test using the device and method of the present invention versus DeLaval Cell Counter. It may be noted that the term, ‘Infected’ includes both subclinical and clinical mastitis—infected cows

TABLE 1
Agreement with DeLaval Cell Counter (%)

| | |
|---|---|
| Healthy | 94 |
| Infected | 86 |

<=500 cells/μL: Healthy
>500 cells/μL: Infected

Overall, the SCC test using the device of the present invention has a sensitivity of 86% and specificity of 94% versus the DeLaval Cell Counter device. Table 2 shows the sensitivity and specificity statistics for the SCC test using the device and method of the present invention for the samples processed at the sites

TABLE 2
Sensitivity and Specificity of Fauna SCC test (%)

| | |
|---|---|
| Sensitivity | 86 |
| Specificity | 94 |
| Positive event | Infected cow (>500 cells/μL) |
| Negative event | Healthy cow (<=500 cells/μL) |

FIG. 7 is a graph that compares the performance of the SCC test using the device and method of the present invention with the gold standard DeLaval Cell Counter (DCC) in terms of linear regression model. The data represents samples across the measuring range from a pilot study from 78 cows. The samples were tested about three to six hours after milking from the cows. The SCC test using the device of the present invention showed good correlation versus the DeLaval Cell Counter with R²=0.88, with a slope of 0.97. Generally, R²>0.8 and a slope >0.9 indicates a good performance of the test method, in this case the method of the present invention, versus the comparator method which in this case used the DeLaval Cell Counter.

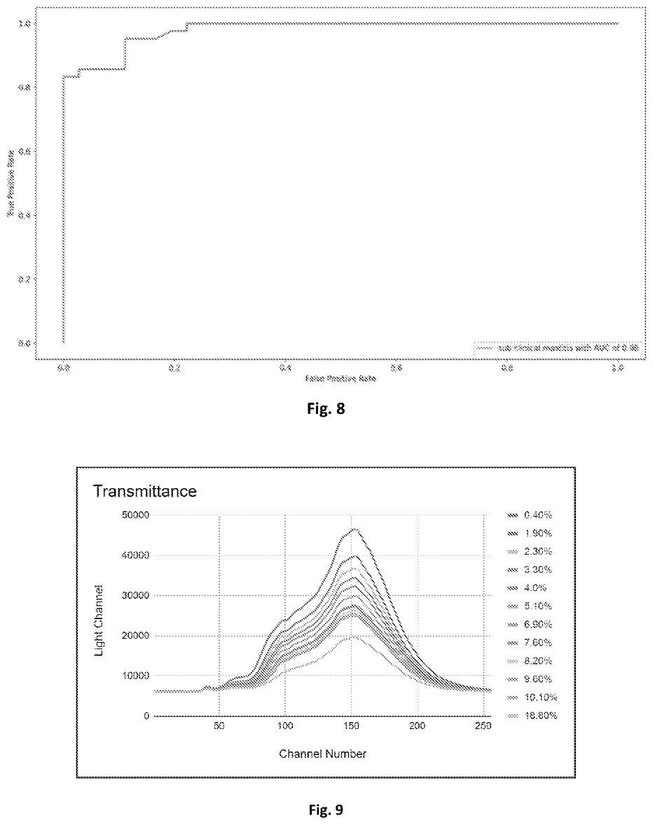

FIG. 8 is a graph that shows the Receiver Operating Characteristic (ROC) curve for the SCC Test using the device and method of the present invention versus the DeLaval Cell Counter. ROC analysis is a useful tool for evaluating the performance of diagnostic tests that classifies subjects into diseased or non-diseased, in this case mastitis-infected or healthy cow. The ROC curve shows the trade-off between sensitivity (or TPR) and specificity (1-FPR). ROC whose curves give closer to the top-left corner indicate a better performance.

In FIG. 8, the AUC is an overall summary of diagnostic accuracy. An AUC >0.5 indicates a good diagnostic accuracy of the test method. In this case, the AUC is 0.98 for sub-clinical mastitis, meaning there is a 98% chance that the SCC Test method of the present invention will be able to distinguish between positive, sub-clinical mastitis cows and negative, healthy cows

FIG. 9 is a graph representing the transmittance data collected by the device of the present invention for cow milk samples with increasing values of milk fat %. FIG. 9 shows the decreasing intensity of the peak with an increase in milk fat %. FIG. 9 show data from cow milk samples having various levels of milk fat % (as measured by a reference analyzer in the lab). The device of the present invention measures the milk fat % on the lines of principles of light scattering method. The cow milk samples are measured using a cuvette placed in an adaptor on the device. Light from the LED passes through the milk sample-filled cuvette and the remaining light transmitted to the optical detector/micro spectrometer sensor, i.e. the transmittance data, is measured by the device of the present invention. The intensity of the data decreases with an increase in the milk fat %, as can be seen in the FIG. 9 graphs i.e. 0.4% to 18.8%.

### Example 2: LDH Test

LDH is an enzyme present in milk when cells are damaged during an udder infection. LDH is correlated to SCC, but is not as easily affected by other conditions such as stress, nutrition, parity, or stage of lactation. LDH levels often rise earlier than somatic cell counts, making it an excellent marker for early detection of udder infections. There are many widely accepted indicators of intramammary infection (IMI) in the dairy animal including somatic cell counting (SCC) and electrical conductivity measurement (EC), but an up-and-coming indicator is LDH. It is the producer-defined herd health management strategy and business need that determines which test of IMI is best suited for the dairy animal and herd. Somatic cell counting is a valuable tool in determining the presence or absence of mastitis in a dairy animal or when screening a bulk tank, but an alternative test to somatic cell counting is the LDH test, a quick dipstick test for the enzyme. Some research suggests that LDH is just as or more effective than SCC at screening for subclinical and clinical mastitis with the benefit of less expense and earlier detection.

The data provided here is from a study that aimed to utilise LDH test strips on the device of the present invention, in order to use LDH as an indicator of udder infection or mastitis (similar to the SCC test). The device and method of the invention estimates the LDH levels in the milk samples based on enzymatic colorimetric reaction and reflectance photometry (similar to the SCC test). In the data provided hereinbelow, the study aimed to understand and correlate the LDH test results with the SCC values from the DeLaval Cell Counter. As shown in Table 3 below, the study involved data collection from 57 cow milk samples in total, out of which 25 samples were healthy and the rest 32 of them were mastitis-infected (based on a SCC cut-off of <500,000 cells/mL categorized as ‘healthy cow’ and >500,000 cells/mL as ‘mastitis-infected cow’).

TABLE 3

| Statistics | LDH test |
|---|---|
| Specificity (in %) | 76 |
| Sensitivity (in %) | 81 |
| Positive Predictive Value (in %) | 81 |
| Negative Predictive Value (in %) | 76 |
| Total number of samples | 57 |
| Healthy samples | 25 |
| Infected samples | 32 |

Cut-offs used
<500,000 cells/ml: Healthy cow
>500,000 cells/mL: Mastitis - infected cow

The study found a considerable relation between the two indicators, with the statistics of sensitivity and specificity standing at 81% and 76% respectively. Thus, the LDH test on the device of the present invention, using the method of the present invention, can be used as an alternative test method for earlier detection of mastitis or other IMI, at a lower cost and shorter testing time. This also shows that the device of the present invention offers a robust platform to integrate and perform various tests for a diverse range of biomarkers in the milk.

The above examples are non-limiting. The invention is defined by the claims that follow:

1.-10. (canceled)

11. A portable device for testing agro-dairy based samples, comprising:
(a) at least one optical detector based on micro-spectroscopy, the detector being optically aligned with at least one test sample and having sensitivity across a wavelength range encompassing reflectance or transmission wavelength of at least one test analyte of the test sample;
(b) at least one adaptor holding:
(i) at least one colorimetric test strip upon which is placed the at least one test sample comprising the at least one test analyte;
(ii) at least one concave shaped holder for holding the at least one test sample, the sample being solid or semi-solid; and
(iii) at least one cuvette for holding the at least one test sample further containing at least one test analyte;
the adaptors for each of (i), (ii) and (iii) fitting into at least one socket on the device;
(c) at least one light emitting diode (LED) light source capable of emitting a range of wavelengths;
(d) a microcontroller for controlling the functioning of electronic components; and
(e) a networking module for data transfer utilizing wireless technology.

12. The device as claimed in claim 11, wherein the optical detector is optically aligned with a reaction pad located on the test strip, wherein the at least one test analyte reacts with a chemical compound present in the reaction pad to produce a specific color, the detector detecting the reflectance of the color.

13. The device as claimed in claim 11, wherein the optical detector is optically aligned with the cuvette to detect a transmission spectrum of the test analyte therein.

14. The device as claimed in claim 11, wherein the adaptor is fused to the concave shaped holder of (b)(ii) forming a single unit.

15. The device as claimed in claim 11, further comprising a storage space within the device to store additional adaptors.

16. A system of testing agro-dairy based samples, comprising:
(a) the portable testing device as claimed in claim 15;
(b) a power source; and
(c) a computing device remotely connected to the portable testing device via the wireless technology of the networking module, the computing device having a computer program for monitoring, recording, storing, or analysis of spectral data, or a combination thereof, wherein the computing device has an electronic processor that executes the computer program to process the spectral data to provide a measurement value for the samples tested.

17. A method of testing agro-dairy based samples using the system as claimed in claim 16, comprising the steps of:
(a) powering on the portable testing device and remotely connecting the device to the computing device;
(b) inputting suitable information into the computer program of the computing device to enable the computer program to instruct the portable testing device to calibrate the LED light sources and the corresponding optical detectors in a specific way;
(c) introducing the at least one test sample into the at least one cuvette, or onto the at least one colorimetric test strip, or onto the at least one concave holder, and inserting the corresponding adaptors holding the cuvette or the test strip or the concave holder into corresponding sockets on the device;
(d) turning on the LED light sources to direct light towards the samples thereby enabling the corresponding optical detectors to receive light reflected from or transmitted through the sample and communicate the same in the form of spectral data to the computing device via the wireless technology;
(e) executing the computer program on the computing device to process the spectral data to provide a measurement value for the sample tested; and
(f) reading a display screen on the remotely connected computing device showing the measured output data of reflectance or transmission of the samples tested.

18. The method as claimed in claim 17, wherein a reference sample or a blank is run prior to step (c).

19. The method as claimed in claim 17, wherein the spectral data obtained corresponds to and is mapped to somatic cell counts or concentrations of fats, solids-not-fats content, lactate dehydrogenase, b-hydroxybutyrate, antibiotic residues and proteins in the sample analyzed.

20. The method as claimed in claim 17, wherein the samples being analyzed are biological fluids.
